(19) United States
(12) Patent Application Publication
(10) Pub. No.: US 2006/0068004 A1
(43) Pub. Date: Mar. 30, 2006

(54) Buccal delivery of sea cucumber tablets

(75) Inventor: Yogendra Shah, Piscataway, NJ (US)

Correspondence Address: Ashok Tankha;Lipton, Weinberger & Husick, 201 No. Jackson Street, P.O. Box 934, Media, PA, 19063, US

(73) Assignee: Aum Nutrapharm Inc., Trenton, NJ (US)

(21) Appl. No.: 10/951,400

(22) Filed: Sep. 28, 2004

(51) Int. Cl. A61K 9/20; A61K 35/12

(52) U.S. Cl. 424/464; 424/522

(57) ABSTRACT

This invention is directed to a composition and method for the buccal delivery of sea cucumber extract. The composition for the buccal delivery of sea cucumber extract comprises a substantially uniform mixture of an effective amount of sea cucumber extract and sugar. In one embodiment of the invention, the sea cucumber extract is approximately 5% to approximately 35% by weight of the sea cucumber tablet formulation. In another embodiment of the invention, the composition for the buccal delivery of sea cucumber tablets comprises a substantially uniform mixture of an effective amount of sea cucumber extract and a sugar and an artificial sweetener. The compression process for the production of the buccal tablets uses lower pressure than conventional tableting pressures. The above dosage units dissolve quickly in the buccal cavity allowing rapid absorption of the sea cucumber tablets through the buccal mucus membrane into the systemic bloodstream.

### BACKGROUND OF THE INVENTION

The present invention relates to a composition and a method for the buccal delivery of sea cucumber extract.

Sea cucumber is an invertebrate animal native to the south China seas. It belongs to the phylum Echinodermata and class Holothuroidea. There are approximately 134 species of sea cucumber, the most common of which are Apostichopus japonicus Acaudina molpadiodides and Thelenota ananas. In this patent application, any species of sea cucumber, alone or in combination with other species will be generically referred to as sea cucumber.

For commercial uses, sea cucumbers are harvested, dried, powdered and the bulk powder product sold as cucumber extract. Sea cucumber extract is used for a wide variety of medicinal purposes including providing immune support and as an antiviral agent. Sea cucumber extract is also indicated for the treatment of human immunodeficiency virus, Parkinson disease and for incontinence. Other medicinal uses of sea cucumber extract are listed extensively in literature.

Active pharmaceutical ingredients are typically administered to a patient by formulating the active pharmaceutical ingredient into dosage forms such as solid dosage products, parenteral solutions, and nasal drops by admixture of the active pharmaceutical ingredient with acceptable excipients and carriers.

A conventional mode of delivery of active pharmaceutical ingredients is the solid dosage route where the tablet is designed to disintegrate in the gastrointestinal tract and release the active pharmaceutical ingredient contained in the drug for absorption into the patient's systemic bloodstream. Where the solid dosage product is designed to be absorbed into the systemic bloodstream in the gastrointestinal tract, the solid dosage form is administered in the form of a tablet or a powder. Where the solid dosage route is a tablet, the blended tablet components are compacted under high compressive pressure to prevent the tablet from disintegrating before it reaches the gastrointestinal tract.

However, for certain active pharmaceutical ingredients or for substances that exhibit therapeutic activity, absorption of the active into the systemic bloodstream is required before the active reaches the gastrointestinal tract to preclude degradation of the active by the acidity and enzymes in the stomach. For such actives, delivery of the active through the conventional tablet dosage form where the tablet is designed to disintegrate in the gastrointestinal tract is not efficacious. Therefore, such actives need to be delivered so that absorption of the active occurs before the active reaches the gastrointestinal tract.

Sea cucumber extract is an example of a product the activity of which is diminished by the acidity or enzymes in the stomach. The efficacy of the sea cucumber extract is enhanced if the active in the sea cucumber extract is absorbed into the systemic blood stream before the active reaches the gastrointestinal tract.

At present, the dosage forms of sea cucumber extract in the market comprise conventional tablets and capsules that are designed to disintegrate in the gastrointestinal tract where a significant part of the active the active undergoes degradation leaving only a part of the active available for absorption into the systemic blood stream.

This invention overcomes the above sea cucumber active degradation problem by use of a delivery format that accomplishes absorption of the active before the active reaches the gastrointestinal tract. The delivery format is a quick dissolving sea cumber extract composition that disintegrates and dissolves rapidly when placed in the oral cavity where the active is released when it comes in contact with saliva. The released active is thereafter transported through the buccal mucosa into the systemic blood circulation system.

Administration of a composition where the active is absorbed through the buccal mucosa in the oral cavity directly into the bloodstream has been given various names such as buccal delivery system, quick dissolve tablets, fast melt tablets, mouth dissolving tablets, fast dissolving tablets, fast-melt tablets, orodisperse tablets, buccal delivery system, orally disintegrating dosage forms, and buccal tablets. In this invention, delivery of the active in sea cucumber extract through the buccal mucosa will be referred to as buccal delivery and the composition for such delivery as a buccal delivery composition.

### SUMMARY OF THE INVENTION

Accordingly, it is an object of the invention to address the aforementioned need in the art by providing a buccal delivery composition for sea cucumber extract.

An aspect of the present invention is to provide a stabilized tablet or powder for the delivery of sea cucumber extract that quickly dissolves in the mouth when it comes in contact with saliva, and thereafter allows the transport of the active in the sea cucumber extract through the buccal mucosa into the circulating bloodstream thereby avoiding the chemical and enzymatic degradation of the active in the stomach and gastrointestinal tract.

A tablet that disintegrates and dissolves quickly on contact with saliva in the mouth generally needs to have low mechanical strength. Therefore, the formulation of the buccal delivery tablet requires a tablet that offers acceptable disintegration and dissolution speed in the oral cavity but also possesses sufficient mechanical strength to withstand disintegration during the course of manufacture and subsequent distribution.

It is still another aspect of the invention to develop a quick dissolving tablet for the buccal delivery of sea cucumber extract by maximizng the porous structure of the tablet matrix by use of compression pressure lower than the compression pressure used in conventional tablet compression, and by use of highly water-soluble excipients in the formulation.

Additional objects and novel features of the invention will be set forth in part in the description which follows and in part will become apparent to those skilled in the art upon examination of the following invention.

### DETAILED DESCRIPTION OF THE INVENTION

Buccal delivery of the sea cucumber extract is achieved by a composition comprising a substantially uniform mixture of at least an effective amount of sea cucumber extract, sugar, or a sugar and an artificial sweetener, and other excipients that are used in the solid dosage art to manufacture tablets and powders.

The sea cucumber extract is milled with sugar, or a sugar and an artificial sweetener, and excipients into a fine dry powder. The mixture may then either be compressed into a tablet, or filled as a powder in an appropriate container such as a blister pack from which the powder may be dispensed.

The sea cucumber extract content of the sea cucumber buccal delivery composition may be increased to any content with a corresponding decrease in the sugar, or sugar and sweetener content. For example, a formulation comprising 5% by weight of sea cucumber extract will contain approximately 93% by weight of sugar and approximately 2% by weight of other excipients used in the solid dosage art such as lubricants to facilitate the ejection of the tablet from the tablet press or provide flowability to the powder, masking flavor, preservatives, dispersants, flavors and the like. Again, by way of example, increasing the sea cucumber extract content in the sea cucumber extract composition to approximately 45% by weight will require the sugar content to be decreased to approximately 53% by weight with the remaining 2% by weight of the formulation comprising other excipients such as lubricants, masking flavor, preservatives, dispersants, flavors and the like.

In another embodiment of the invention, the sea cucumber extract buccal delivery composition contains at least an effective amount of a sea cucumber extract, a sugar and a sweetener. The sea cucumber extract content of the sea cucumber tablet or powder formulation may be increased to any content with a corresponding adjustment in the sugar and sweetener content. In addition to the sugar and sweetener, the formulation contains small quantities of excipients known in the solid dosage manufacturing art such as lubricants, masking flavor, preservatives, dispersants, flavors and the like.

Where the buccal delivery composition is administered by tablets, the compression pressures used for tableting are relatively lower than compression pressures used for the manufacture conventional tablets, in order to obtain a tablet that provides higher porosity and faster disintegration in the mouth than a conventionally manufactured tablet. The buccal delivery tablets under this invention disintegrate in the mouth in approximately 10 seconds to approximately 30 seconds. Where the buccal delivery of the sea cucumber extract is administered via a powder dosage form, the dissolution time in the mouth is approximately of the same order of magnitude.

A feature of the buccal delivery composition and dosage form of the present invention is the rapid disintegration and release of the active in the oral cavity when it comes in contact with saliva, the subsequent transport of the active through the buccal mucous lining in the oral cavity and absorption of the active into the systemic blood circulation system, thereby precluding exposure of the active to the acidic environment in the stomach and gastrointestinal tract.

The fast dissolving property of the tablet or powder is attributable to the quick ingress of saliva into the sugar, or sugar and sweetener matrix resulting in its rapid disintegration. Hence, the basic approach that was used to develop the quick dissolving sea cucumber tablet was to maximize the porous structure of the tablet matrix by use of lower compression pressure than the compression pressure used for conventional tablet compression, and use of highly water-soluble excipients in the formulation. Such tablets are less compact than conventional compressed tablets and possess a more porous structure that allows rapid dissolution when the tablet comes in contact with saliva in the oral cavity.

An exemplary formulation adapted for buccal delivery of sea cucumber extract according to this invention includes at least three components: (1) an effective amount of the sea cucumber extract; (2) a quick dissolving binder; and (3) excipients that are well known in the solid dosage art.

The active in the sea cucumber buccal delivery dosage form under this invention is present in the sea cucumber extract. This active may be also be synthesized, or extracted from a naturally occurring source such as sea cucumbers.

The binder used in the buccal formulation of the present invention is a quick dissolving sugar, a polyol, sucrose, glucose, dextrose, fructose, isomalt, lactitol, maltitol, maltose, mannitol, sorbitol, starch hydrolysate, and xylitol that display high aqueous solubility and impart taste masking and a pleasant mouth-feel. Lactose and mannitol exhibit a high dissolution rate but low moldability. Maltose and maltitol exhibit higher moldability but a lower dissolution rate. The lubricant used in the formulation is selected from any of the conventionally used lubricants for the manufacture of solid dosage forms such as silicon dioxide, magnesium stearate, etc.

In another embodiment of the formulation, sea cucumber quick dissolving tablets or powders were formulated using artificial sweeteners and a filler as excipients. The artificial sweeteners need to be quick dissolving sweeteners such as stevia, sacchirin, neohesperidine, aspartame, etc. Other additives that may be used in the formulation of the sea cucumber tablet include preservatives, dispersants, lubricants and flavors.

The dosage of the sea cucumber extract buccal composition will vary depending on factors such as severity of the condition being treated, age, body weight of the patient, diet, etc. As a general guide, patients with a body weight in the range of 60-90 kg would ingest about 1 to 2 tablets, 3 times a day of the sea cucumber extract tablet containing 100 mg of the sea cucumber extract /tablet. One benefit of the buccal sea cucumber extract medications as sea cucumber extract is a dietary supplement.

The raw materials used for the manufacture of sea cucumber extract tablets and powders were sourced from the following vendors:
Sea cucumber extract: IRMA Co., Edison, N.J.
Sugar: Domino's Specialty Ingredients, Baltimore, Md.
Natural peppermint flavor: Mother Murphy's Lab, Greensboro, N.C.
Magnesium stearate USP: Ruger Inc., Newark, N.J.
Stevia: QBI, So. Plainfield, N.J.
Silicon dioxide: Cabot Corp., Tuscola, Ill.
Masking flavor: Mother Murphy's Lab, Greensboro, N.C.
Vitamin C: Graymor Inc., Elizabeth, N.J.
Mannitol and other sweeteners: SPI, Wilmington, Del.
   Examples of sea cucumber buccal delivery compositions and the method for making the dosage forms are provided below.

### EXAMPLE 1

Buccal sea cucumber tablets weighing approximately 500 mg each were manufactured using the composition tabulated below:

| Ingredient | Weight, mg | % by weight |
|---|---|---|
| Sea cucumber extract | 20.00 | 5 |
| Sugar | 368.00 | 92 |
| Natural peppermint flavor | 2.00 | 0.5 |
| Magnesium Stearate USP | 4.40 | 1.1 |
| Stevia, SE 90% | 0.72 | 0.144 |
| Silicon dioxide | 2.41 | 0.482 |
| Masking flavor | 1.93 | 0.386 |
| Vitamin C | 1.45 | 0.290 |
| Total | 500.00 | 100.00 |

All components listed above were mixed in a blender. The blend was compressed in a conventional rotary tablet press using flat face beveled edge tooling of 0.5 inch diameter. Tablets with a hardness of 4-8 kilopascals were produced. Alternatively, the blended components may be processed into a container from which the blend may be dispensed.

### EXAMPLE 2

In essentially the same way as described in example 1 above, sea cucumber buccal delivery tablets were manufactured with the composition shown below:

| Ingredient | Weight, mg | % by weight |
|---|---|---|
| Sea cucumber extract | 100 | 20.00 |
| Sugar | 382.16 | 76.4 |
| Natural peppermint flavor | 8.92 | 1.88 |
| Magnesium stearate USP | 2.41 | 0.48 |
| Stevia, SE 90% | 0.72 | 0.14 |
| Silicon dioxide | 2.41 | 0.48 |
| Masking flavor | 1.93 | 0.38 |
| Vitamin C | 1.45 | 0.29 |
| Total | 500.00 | 100.00 |

Tablets with the following characteristics were produced:
Tablet diameter: 12.5 mm
Tablet hardness: 4 to 8 kilopascals
Tablet thickness: 2 to 5 mm
   Alternatively, the blended components may be processed into a container from which the blend may be dispensed.

### EXAMPLE 3

In essentially the same way as described in example 1 above, sea cucumber buccal delivery tablets were manufactured with the composition shown below:

| Ingredient | Weight, mg | % by weight |
|---|---|---|
| Sea cucumber extract | 338 | 35.00 |
| Sugar | 612 | 63.14 |
| Flavor | 9 | 0.93 |
| Magnesium stearate | 3 | 0.31 |
| Masking flavor | 3 | 0.31 |
| Vitamin C | 3 | 0.31 |
| Total | 968 | 100.00 |

Tablets with the following characteristics were produced:
Tablet diameter: 12.5 mm
Tablet hardness: 4 to 8 kilopascals
Thickness: 5 to 6 mm
Tablet weight range: 920 to 1015 mg
   Alternatively, the blended components may be processed into a container from which the blend may be dispensed.

### EXAMPLE 4

In essentially the same way as described in example 1 above, sea cucumber buccal delivery tablets were manufactured with the composition shown below:

| Ingredient | Weight, mg | % by weight |
|---|---|---|
| Sea cucumber extract | 100.00 | 19.68 |
| Sucrose | 300.00 | 59.05 |
| Xylitol | 100.00 | 19.68 |
| Stevia | 3 | 0.60 |
| Aspartame | 1 | 0.20 |
| Lubricant | 4 | 0.79 |
| Total | 508 | 100.00 |

Tablets with the following characteristics were produced:
Tablet diameter: 12.5 mm
Tablet hardness: 4 to 8 kilopascals
Thickness: 2 to 5 mm
Tablet weight range: 483 to 533 mg
   Alternatively, the blended components may be processed into a container from which the blend may be dispensed.

### EXAMPLE 5

In essentially the same way as described in example 1 above, sea cucumber buccal delivery tablets were manufactured with the composition shown below:

| Ingredient | Weight, mg | % by weight |
|---|---|---|
| Sea cucumber extract | 500 | 24.70 |
| Mannitol, xylitol, or sugar | 1500 | 74.26 |
| Magnesium stearate | 20 | 0.99 |
| Total | 2020 | 100.00 |

Tablets with the following characteristics were produced:
Tablet diameter: 20 mm
Tablet thickness: 4 to 8 mm
Tablet hardness: 4 to 8 kilopascals
Tablet weight range: 1915 to 2115 mg
   Alternatively, the blended components may be processed into a container from which the blend may be dispensed.

### EXAMPLE 6

In essentially the same way as described in example 1 above, sea cucumber buccal delivery tablets were manufactured with the composition shown below

| Ingredient | Weight, mg | % by weight |
|---|---|---|
| Sea cucumber extract | 500 | 19.88 |
| Sugar | 1500 | 59.64 |
| Mannitol | 500 | 19.88 |
| Magnesium stearate | 15 | 0.60 |
| Total | 2515 | 100.00 |

Tablets with the following characteristics were produced:
Tablet diameter: 20 mm
Tablet hardness: 4 to 8 kilopascals
Tablet thickness: 4 to 8 mm
Tablet weight range: 2389 to 2640 mg
   Alternatively, the blended components may be processed into a container from which the blend may be dispensed

### EXAMPLE 7

In essentially the same way as described in example 1 above, sea cucumber buccal delivery tablets were manufactured with the composition shown below:

| Ingredient | Weight, mg | % by weight |
|---|---|---|
| Sea cucumber extract | 100 | 24.70 |
| Sugar | 300 | 74.07 |
| Silicon dioxide | 5 | 1.23 |
| Total | 405 | 100.00 |

Tablets with the following characteristics were produced:
Tablet diameter: 12.5 mm
Tablet hardness: 4 to 8 kilopascals
Thickness: 2 to 5 mm
Tablet weight range: 385 to 425 mg
   Alternatively, the blended components may be processed into a container from which the blend may be dispensed

It is to be understood that while the invention has been described in conjunction with the preferred specific embodiments thereof, that the forgoing description as well as the examples that follow are intended to illustrate and not limit the scope of the invention. Other aspects, advantages and modifications within the scope of the invention will be apparent to those skilled in the art to which the invention pertains.

What is claimed is:

1. A composition for the buccal delivery of sea cucumber extract comprising a substantially uniform mixture of an effective amount of sea cucumber extract and sugar.

2. The composition of claim 1 wherein the sea cucumber extract is approximately 5% to approximately 35% by weight of the sea cucumber buccal delivery composition.

3. The composition of claim 1 wherein the sugar is selected from the group consisting of a polyol, sugar, sucrose, glucose, dextrose, fructose, isomalt, lactitol, maltitol, maltose, mannitol, sorbitol, starch hydrolysate, and xylitol

4. The composition of claim 1 in a tablet dosage form.

5. The composition of claim 1 in a powder dosage form.

6. The composition of claim 4 where the tablet dissolves in the mouth in approximately 10 seconds to approximately 30 seconds.

7. A composition for the buccal delivery of sea cucumber extract comprising a substantially uniform mixture of an effective amount of sea cucumber extract, a sugar and a sweetener.

8. A composition according to claim 7 comprising approximately 5% to approximately 35% sea cucumber extract and approximately 0.2% to approximately 25% of a sweetener.

9. The composition according to claim 7 wherein the sweetener is a natural sweetener or an artificial sweetener.

10. A composition according to claim 7 wherein the artificial sweetener is selected from group consisting of stevia, sacchirin, neohesperidine and aspartame.

11. The composition of claim 7 where the tablet disintegrates in the mouth in approximately 10 seconds to approximately 30 seconds.

12. The composition of claim 7 where the dosage form is a tablet.

13. The composition of claim 7 where the dosage form is a powder.

14. A compression process for producing a sea cucumber buccal tablet comprising the steps of:
(a) mixing the sea cucumber formulation comprising sea cucumber extract and excipients,
(b) milling the formulation, and
(c) compacting the formulation into tablets in a tablet press.

15. The compression process of claim 14 wherein the tablet press compression pressure used is about 4 to about 8 kilopascals.
